(19) United States
(12) Patent Application Publication
(10) Pub. No.: US 2022/0031215 A1
(43) Pub. Date: Feb. 3, 2022

(54) ELECTRODE

(71) Applicant: LEONH. LANG, Innsbruck (AT)

(72) Inventor: Thomas KUPSA, Innsbruck (AT)

(21) Appl. No.: 17/502,749

(22) Filed: Oct. 15, 2021

Related U.S. Application Data
(63) Continuation of application No. PCT/AT2020/060137, filed on Apr. 2, 2020.

(30) Foreign Application Priority Data
Apr. 16, 2019 (AT) A 50344/2019

(51) Int. Cl. A61B 5/265; A61B 5/268; A61B 5/259

(52) U.S. Cl. CPC A61B 5/265; A61B 5/268; A61B 2562/125; A61B 2562/227; A61B 2562/0215; A61B 5/259

(57) ABSTRACT

An electrode includes an electrically non-conductive support. A protruding, electrically conductive attachment element with an attachment site for the releasable attachment of a signal conductor is on an upper face of the support. A conductor is on the underside of the support, and is electrically connected to the attachment element and to a contact medium. The conductor and the contact medium are arranged on the underside of the support. An imaginary normal to the support, running through the attachment element, runs through the conductor or a recess in the conductor and through the contact medium or a recess in the contact medium. The conductor has on its surface a redox pair suitable for the depolarization of the electrode. The attachment element has a projection reaching through the support with a widened region at its end, and the conductor is arranged between the widened region and the support.

### BACKGROUND OF THE INVENTION

The invention concerns an electrode and a method of producing an electrode.

Medical skin electrodes of that kind can be used as measurement electrodes which derive electrical signals from the human body. They can however also be used as therapy electrodes to feed currents to the human body. For that purpose the electrodes are glued on to the skin and on their underside generally have an electrically conducting gel or another electrical contact medium which is galvanically in contact with a connecting element of the electrode. An electrical signal conductor can be connected to that connecting element, by way of which conductor currents can be taken from the electrode or fed to the electrode.

One type of electrode has at the top side facing away from the skin a projecting electrically conducting connecting element with a generally substantially ball head-shaped connecting location to which a neck is connected.

In the previous construction of electrodes of that type the connecting element is of a two-part structure. The upper part (upper knob or stud) serves as a contact and anchor element for commercially usual signal conductors, for example ECG lines. Substantially beneath the carrier, that is to say on the side facing towards the skin, there is a lower knob (eyelet) which serves for the transfer of electrical potentials directly from the gel (contact medium) or for transmission to the gel. In that case the eyelet is connected both mechanically and electrically to the stud, more specifically generally by riveting of the two parts, in such a way that the carrier material of the electrode is fixedly clamped between a holding region of the stud, that projects laterally like a flange, and a likewise holding region of the eyelet. Such a construction affords on the one hand a good mechanical hold for the connecting element to the carrier of the electrode while on the other hand it makes it possible to make the eyelet from materials which have favorable electrical properties for a signal electrode, for example for that purpose it can be coated with silver, in which case the silver coating can in turn be covered over its entire area or at least in a partial region which is in contact with the gel with a layer of silver/silver chloride (Ag/AgCl).

The electrodes in accordance with the state of the art however are costly—in that respect just minor differences in price are significant in relation to mass-produced articles of that kind.

In addition the layer comprising for example silver/silver chloride (Ag/AgCl) in the case of electrodes in the state of the art are in contact with the contact medium over the full area. That has the result that from the beginning (first contact on the part of the contact medium with the silver/silver chloride layer) the silver/silver chloride layer is attacked by the contact medium. Therefore the silver is converted into silver chloride by the contact medium at the entire surface area of the silver/silver chloride layer. Therefore a comparatively large amount of silver has to be provided to guarantee proper functionality of the electrode. That in turn contributes to the high costs of electrodes in accordance with the state of the art.

### SUMMARY OF THE INVENTION

The object of the invention therefore is to provide an improved electrode which in particular avoids the above-mentioned problems, and a method of producing such an electrode.

In that way, it is possible for the expensive and elaborate operation of coating the entire eyelet (connecting element) with for example silver/silver chloride to be replaced by the substantially less expensive conductor which is easy to produce.

Particularly preferably, the side of the conductor, that faces towards the contact medium, is covered partially or completely by the connecting element.

As a result, only a small part or only the edge layer of the silver/silver chloride layer is in contact with the contact medium. The result of this is that only that small part or only the edge layer of the silver/silver chloride layer can be attacked by the contact medium and thus less silver can be converted into silver chloride in the same time. Conversion takes place only slowly from the regions of the silver/silver chloride layer, that are in contact, to the covered regions of the silver/silver chloride layer. It is therefore possible to reduce the amount of silver in the silver/silver chloride layer and thus save on further costs.

The connecting element can comprise a single part which has the connecting location for releasably connecting a signal line.

However, the connecting element can also comprise at least two parts, wherein one of the two parts has the connecting location for releasable connection of a signal line.

The connecting element itself can, in that case, comprise a plurality of materials, for example nickel-plated brass or a plastic doped with conductive material (in particular carbon fibers).

A particularly preferred configuration of the connecting element is one in which it is of such a configuration that the connecting element has a substantially ball-shaped head, an adjoining neck of reduced diameter, a holding region which adjoins the end of the neck and which projects laterally in a flange shape and at least one projection adjoining the holding region.

In the case of a one-part connecting element, the projection is passed through an opening in the carrier (preferably without making lateral contact therewith) while the holding region projecting laterally in a flange shape bears against the top side of the carrier. The holding region of enlarged diameter which projects laterally in a flange shape holds the connecting element firmly and securely to the carrier material even under high pressure loadings.

The deformed enlarged region of the projection of the holding element bears against the underside of the carrier, that faces towards the skin, or against the conductor, and thus ensures a good hold for the connecting element on the carrier, even in the event of pressure loadings on the connecting element.

In a two-part connecting element, the projection is passed through an opening in the carrier (preferably without lateral contact therewith) while the holding region which projects laterally in a flange shape bears against the underside (or top side) of the carrier. The second part of the connecting element is then arranged on the projection and bears on the top side (or underside) of the carrier.

In a further embodiment of the invention, the at least one projection is in the form of a spike which narrows in a direction opposite to the holding region. In that way, it is possible for the connecting element to be introduced into the carrier or conductor without previously making a through opening through the conductor and the carrier. That, therefore, saves on a working step.

High demands are not made on the electrical properties of the connecting element in the case of the subject of the invention. It can therefore comprise inexpensive material, for example a simple metal sheet. More specifically the connecting element does not need to have any particular electrical properties for only the conductor which is in contact with the electrical contact medium can have those electrical properties which are advantageous in terms of bioelectrodes.

In that respect, that conductor can basically be of any desired geometry, in preferred embodiments of the invention however the conductor can be in the form of a rotationally symmetrical or substantially cuboidal conductor plate. That conductor plate can project at least partially over the deformed enlarged region.

In order to achieve a low level of noise and depolarization in the case of defibrillation in respect of an electrode redox couples are currently used. They can be oxidized or reduced and in that case receive or give off at least one electron. The most widely varying substances are used for such depolarization at the present time. Silver/silver chloride and tin/tin chloride are most frequently used. It will be appreciated, however, that all redox couples which permit depolarization of the electrode are possible for the present invention. In that respect, the redox couples can be actively added or possibly generated in situ by reactions.

As, for example, silver/silver chloride is a relatively costly substance, it is sufficient if in accordance with a further aspect of the invention the conductor preferably has at one side an electrically conducting material which is galvanically joined to the connecting element and to the contact medium.

Further costs can be saved by the measure of providing the conductor, preferably at one side, with an electrically conducting material. More specifically the actual conductor can use inexpensive materials like for example metal or plastic while a second electrically conducting material like for example silver/silver chloride can be used at the transition region to the electrical contact medium (in particular gel), that is critical for the desirable electrical properties of the bioelectrode. It is sufficient if such material is only locally present in that region.

In particular, the conductor can comprise a plastic film provided with an electrically conducting material.

Overall, the basic concept of the invention is to provide the connecting element for the signal conductor in such a fashion that it is well anchored in the electrode while the electrical properties are a less important consideration and thus inexpensive materials can be employed.

On the other hand, the more expensive materials which are provided for the advantageous electrical signal line can be used only in the electrical critical region at the transition to the electrical contact medium (gel). The conductor performs that function. Stated in quite brief terms, it would be said that the electrically conducting connecting element, apart from the basic property of electrical conduction, is primarily responsible for the “mechanics”. The reverse applies in respect of the conductor: it does not need to fulfill any particular mechanical properties and it is only in the region of the transitional location to the electrical contact medium (gel) that it comprises materials which are desirable for that purpose. In that respect, the conductor is responsible for the “electrics” without any particular mechanical functions.

### BRIEF DESCRIPTION OF THE DRAWINGS

Further advantages and details of the invention are described by means of the specific description hereinafter. In the drawings:

FIG. 1 shows a diagrammatic view from below (later the side facing towards the skin) of the production steps of an embodiment of an electrode according to the invention to the finished electrode,

FIG. 2 shows a diagrammatic plan view of the production steps of an embodiment of an electrode according to the invention to the finished electrode, with only a part of the process steps being shown as a plan view,

FIG. 3 shows a sequence of the sections along line A-A in FIG. 1, where the view is to be interpreted as a diagrammatic view for better visualization,

FIG. 4 shows a diagrammatic view from below (later the side facing towards the skin) of the production steps of a further embodiment of an electrode according to the invention to the finished electrode,

FIG. 5 shows a diagrammatic plan view of the production steps of a further embodiment of an electrode according to the invention to the finished electrode, with only a part of the process steps being shown as a plan view,

FIG. 6 shows a sequence of the sections along line A-A in FIG. 7, where the view is to be interpreted as a diagrammatic view for better visualization,

FIG. 7 shows a diagrammatic view from below of an embodiment of a carrier according to the invention with an adhesive layer,

FIG. 8 shows a diagrammatic view from below of a further embodiment of a carrier according to the invention with an adhesive layer,

FIG. 9a shows a diagrammatic side view of an embodiment of a connecting element according to the invention,

FIG. 9b shows a diagrammatic plan view of an embodiment of a connecting element according to the invention,

FIG. 10a shows a diagrammatic side view of an anchoring process of a connecting element according to the invention in a carrier,

FIG. 10b shows a diagrammatic plan view (later the side facing away from the skin) of an anchoring process of a connecting element according to the invention in a carrier,

FIG. 11a shows a diagrammatic view of a further embodiment of a connecting element according to the invention,

FIG. 11b shows a diagrammatic view of a further embodiment of a connecting element according to the invention,

FIG. 12a shows a diagrammatic side view of a further embodiment of a connecting element according to the invention,

FIG. 12b shows a diagrammatic plan view of a further embodiment of a connecting element according to the invention,

FIG. 13a shows a diagrammatic side view of a further embodiment of a connecting element according to the invention,

FIG. 13b shows a diagrammatic plan view of a further embodiment of a connecting element according to the invention,

FIG. 14a shows a diagrammatic side view of a further embodiment of a connecting element according to the invention,

FIG. 14b shows a diagrammatic plan view of a further embodiment of a connecting element according to the invention,

FIG. 15 shows an exploded view of a further embodiment with a two-part connecting element,

FIG. 16 shows a diagrammatic view from below (later the side facing towards the skin) of the production steps of an embodiment (two-part connecting element) of an electrode according to the invention to the finished electrode,

FIG. 17 shows a diagrammatic plan view of the production steps of an embodiment according to the invention to the finished electrode, with only a part of the process steps being shown in a plan view, and

FIG. 18 shows a sequence of the sections along line A-A in FIG. 15, wherein the view is to be interpreted as a diagrammatic view for better visualization.

### DETAILED DESCRIPTION OF THE INVENTION

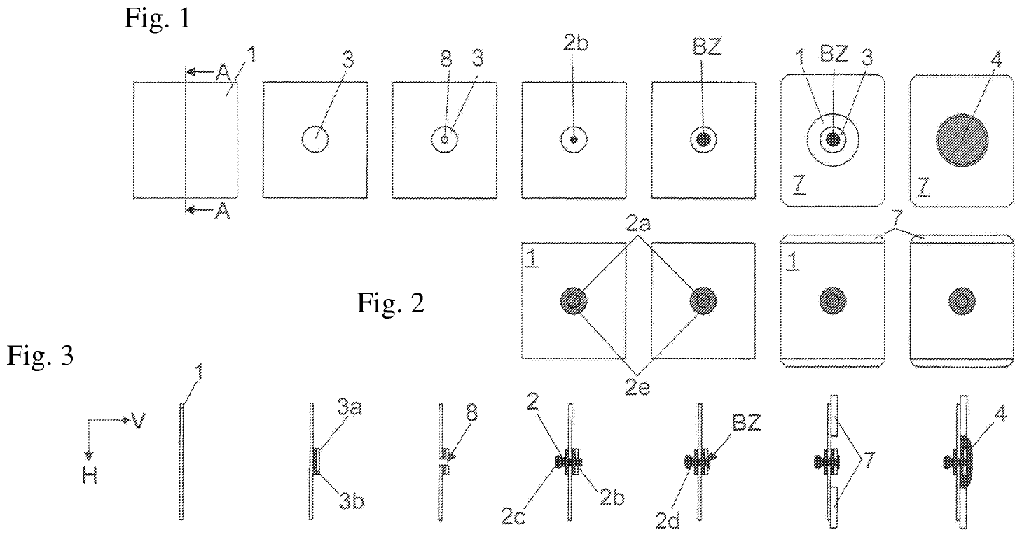

With reference to FIGS. 1 through 3 the procedure of the method for the production of an embodiment of an electrode according to the invention for application to the human skin is now described in greater detail.

The basic starting point is an electrically non-conducting carrier 1. The carrier material serves for anchoring the electrical components of the electrode. It can comprise for example a (flexible) film (for example of PET or TPU) which on the underside facing upwardly in the drawing of FIG. 1 is completely or partially coated with an adhesive which for example can be self-adhesive (pressure sensitive adhesive) or thermoactivatable (hot melt).

Now in a next step a rotationally symmetrical conductor 3 is fixed on that carrier material, preferably by adhesive or by being printed thereon. In accordance with a preferred variant of the invention the conductor has two differently electrically conducting materials or an electrically non-conducting material 3b and an electrically conducting material 3a, wherein the electrically conducting material 3a or one of the two electrically conducting materials is later galvanically connected to the electrical connecting element 2 and to the contact medium 4 (gel).

The illustrated embodiment involves a circular conductor 3 of a plastic film, which is shown in black or gray. The conductor 3 however can also comprise a metal or a conductive plastic doped with carbon fibers.

In the region of the later contact location with the electrical contact medium 4 (gel) that conductor 3 is coated with a layer 3a of for example silver/silver chloride or tin/tin chloride or another redox couple.

In a further step an opening 8 is now provided through the electrical conductor 3 and the carrier 1. That can be done by stamping. The connecting element 2 which has a projection 2b which projects beyond the underside of the carrier 1 and the conductor 3 is then introduced.

In the illustrated embodiment adjoining the substantially ball-shaped head 2c the connecting element 2 has a neck 2d of reduced diameter, which is adjoined by a holding region 2e projecting laterally in a flange shape, and a projection 2b.

Overall the laterally projecting flange-shaped holding region 2e is of a substantially plate-shaped configuration. It is responsible for distribution and transmission of pressure forces applied to the connecting element 2, to the carrier 1.

When using a connecting element 2 which comprises a single part which on the one hand is connected to the electrical conductor 3 and which on the other hand has the connecting location 2a for releasable connection of a signal conductor (not shown here) inexpensive manufacture of the electrode is possible in that way because the generally cost-intensive eyelet (underneath knob) can be omitted. The one-part configuration of the connecting element is sufficient for mechanical anchoring.

The demands made in terms of the electrical properties are low. In that way it is possible to use simple structures like for example a deep-drawn metal part as the connecting element 2. The somewhat more difficult electrical functions are therefore implemented here not by the otherwise usual eyelet but the conductor 3 which is joined to the electrical contact medium 4 (gel) which is later applied.

This therefore involves separation of the functions. Apart from the basic property of being electrically conducting the electrical connecting element 2 is substantially responsible for the mechanical hold in the electrode while the conductor 3 is substantially freed of mechanical tasks. That makes it possible to adopt a favorable material. In particular it is possible to provide more costly materials—which are favorable from the electrical point of view—only where (location 3a) contact with the gel later occurs.

As already mentioned the electrically conducting connecting element 2 can comprise a deep-drawn metal sheet. It is then at least partially hollow in its interior. It can however also comprise a conductive plastic, for example ABS, which is doped with conductive carbon fibers.

More desirably the connecting element is of a substantially rotationally symmetrical configuration. Other variants are also possible.

In order to fix the electrical connecting element 2 definitively in the electrode and in particular also to secure it against tensile loadings a next step provides for deforming the projection 2b in such a way as to produce a deformed enlarged region BZ.

Deformation of the projection 2b can be effected in that case by fusing, beading over, spreading or bending over. It is however also possible to use any other suitable method.

The deformation of the projection 2b provides that a galvanic connection is made between the connecting element 2 and the conducting material 3a of the conductor by way of the deformed enlarged region BZ while on the other hand mechanical fixing of the connecting element 2 to the carrier 1 is effected by means of positively locking and/or force-locking relationship.

A plaster layer 7 is now applied to the underside of the carrier 1, in particular by adhesive, wherein the plaster layer can preferably be stuck on the skin by means of a patient-side coating of biocompatible plastic in order to fix the electrode.

In that respect it is also possible for the plaster layer to be glued to the carrier 1 by way of a layer applied to the plaster layer and comprising pressure-sensitive adhesive or a thermoactivatable adhesive.

The plaster material ultimately serves to fix the electrode on the patient skin. Suitable plaster materials can comprise for example a film (for example PE), a foam band (for example PE foam) or non-woven materials. The plaster materials are usually coated on the patient side with a biocompatible adhesive.

In the last step in the production of the electrode shown in FIGS. 1 through 3 the electrical contact medium 4 is introduced into a recess provided for same in the plaster material 7. The electrical contact medium 4 permits the (preferably ion-based) conduction of body-generated electrical potentials or device-generated measurement or stimulation currents from the body surface (skin) to the electrical connecting element 2 and vice-versa. The contact medium can for example comprise a gel which is doped with chlorides and which is present either in a more or less liquid form (more or less gelled) or in the form of a cross-linked polymer matrix (hydrogel). It is however also possible to create the electrical contact medium 4 with other means, for example in the form of conductive adhesives or in the form of sponge filled with saline solution.

At any event the electrical contact medium 4, as the last step in FIGS. 1 through 3 shows, is introduced into the recess in the plaster material 7. It contacts therein the electrically conducting material 3a (in particular silver/silver chloride).

The cooperation of the electrically conducting material 3a, in particular the coating with silver/silver chloride or another suitable material on the one hand and the material of the electrically conducting contact medium 4 on the other hand makes it possible to achieve favorable electrical properties of the electrode like for example noise-free signal transmission or depolarizing effects, in which case the use of the relatively costly electrically conducting material 3a of the conductor 3 can remain restricted to that region in which contact with the contact medium 4 occurs. That further reduces the costs.

Overall in the production shown in FIGS. 1 through 3 there is a “central” electrode in which the connecting element 2 and the contact medium 4 (gel) are arranged directly above each other.

The method steps which are essential for the embodiment shown in FIGS. 1 through 3 are as follows:
arranging, preferably gluing or printing, a conductor (3) on the underside, towards the skin, of an electrically non-conducting carrier (1),
introducing a connecting element (2) through the carrier (1) in such a way that the projection (2b) of the connecting element (2) projects on the underside or the top side of the carrier (1) and the connecting element (2) bears against the top side or the underside of the carrier (1)—preferably with a laterally projecting plate-shaped holding region (2e), and
anchoring the connecting element (2) in the carrier (1) in such a way that an electrically conductive connection is made between the connecting element (2) and the conductor (3) and a mechanical fixing of the connecting element (2) is made on the carrier (1).

Finally the following steps are then also implemented to finish the electrode:
applying—preferably gluing—a plaster layer (7) which is adhesive on the skin side to the carrier (1), and
introducing an electrical contact medium (4)—preferably a gel—into a recess in the plaster layer (7) in such a way that the subjacent conductor (3) is contacted.

The deformed enlarged region BZ can also not be circular but of a lamellar configuration. The deformed enlarged region BZ can basically be of any desired shape.

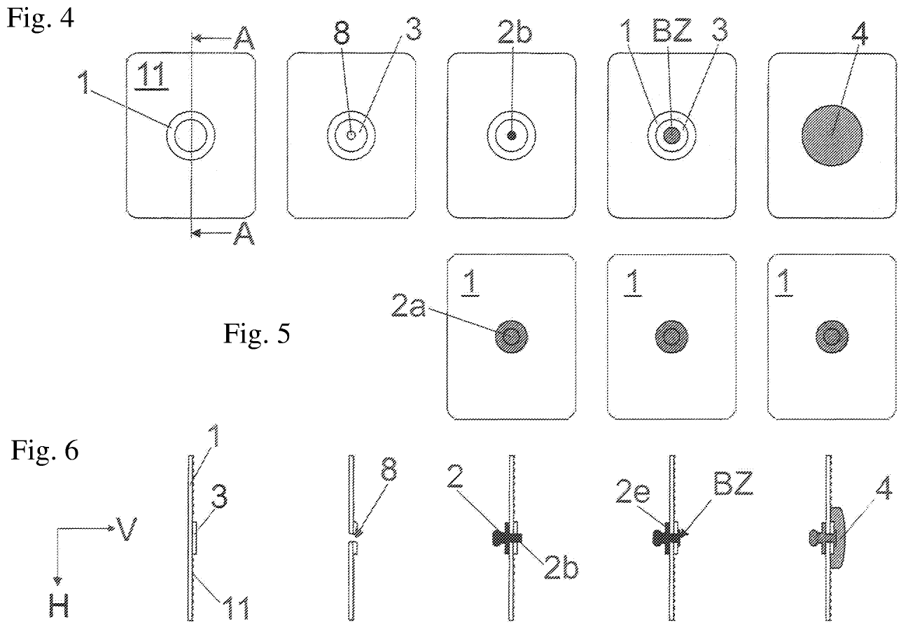

In the embodiment shown in FIGS. 4 through 6 most of the method steps are the same as those in FIGS. 1 through 3, for which reason identical references also denote the same parts.

The difference is substantially that there is provided on the carrier 1 a biocompatible adhesive layer 11 for attaching the electrode to the skin of a patient. The plaster layer 7 can thus be eliminated and a further process step is saved.

In this case the adhesive layer 11 can be applied prior to or after application of the conductor 3 to the carrier 1 or the adhesive layer 11 is already provided on the starting material of the carrier 1.

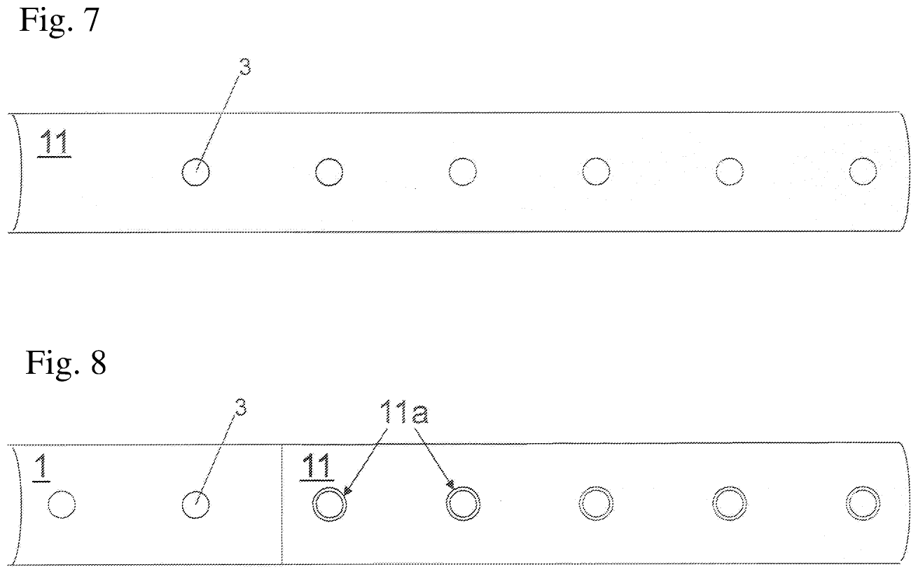

The above-mentioned variants for applying the adhesive 11 are shown in FIGS. 7 and 8.

In FIG. 7 the adhesive 11 is already present on the carrier 1 or is applied prior to application of the conductor 3. The conductor 3 is then applied to the adhesive layer 11. In that case the conductor 3 can be held by the adhesive layer 11 whereby the conductor 3 does not have to be additionally glued to the carrier 1.

In FIG. 8 the conductor 3 is applied to the carrier 1 and then the adhesive 11 is applied to the carrier 1. In this case there is provided an opening 11a so that the conductor 3 is not covered by the adhesive 1.

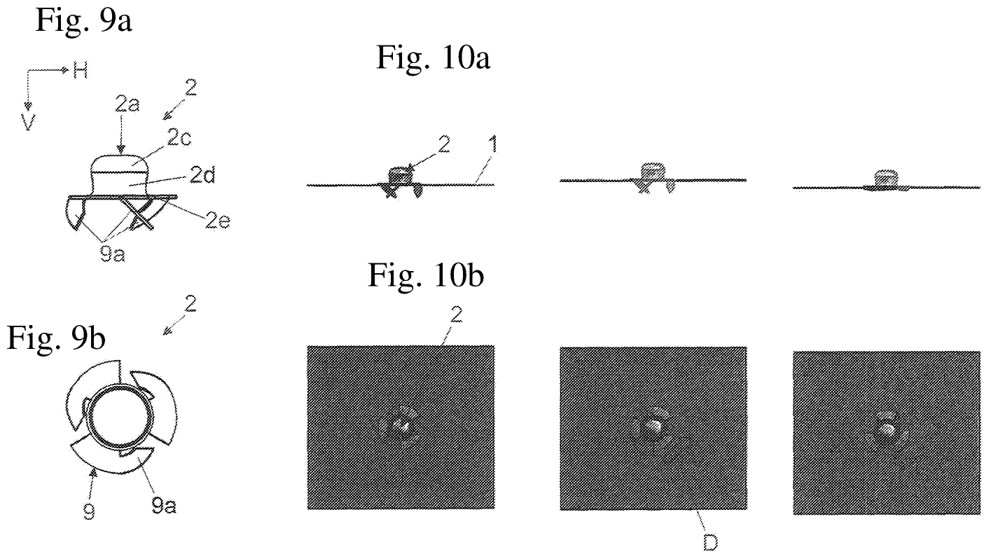

FIGS. 9a and 9b show an embodiment of a connecting element 2 according to the invention. It can be seen that the connecting element 2 has wing segments 9 which form both the projection and also the holding region of the connecting element 2. The wing segment portions 9a which are inclined with respect to a horizontal position H can be of the same or differing lengths.

It is also conceivable that the wing segments 9 are of a sharp-edged configuration at least portion-wise to facilitate penetrating a carrier 1 and a conductor 3.

FIGS. 10a and 10b show diagrammatic views of an anchoring procedure for a connecting element according to the invention in a carrier, with a connecting element 2 as shown in FIGS. 9a and 9b.

For that purpose in a first step the connecting element 2 is pushed from a top side of a carrier 1, that later faces away from the skin, through the carrier 1 and the conductor 3 (not shown) which is attached to the underside of the carrier 1. This means that the connecting element 2 penetrates the carrier 1 and the conductor 3 with the wing segment portions 9a.

In a next step the connecting element 2 is rotated in a direction D. That provides for better anchorage of the connecting element 2 in the carrier 1.

In a last step the wing segment portions 9a are bent up in the direction of the underside of the carrier 1 beyond a horizontal position H whereby the carrier 1 and the conductor 3 are clamped. This also ensures an electrical connection of the connecting element 2 to the conductor 3 and a mechanical fixing of the connecting element 2 on the carrier 1. It will be appreciated however that it is also possible for the wing segment portions to be only bent up until they are in a horizontal position H.

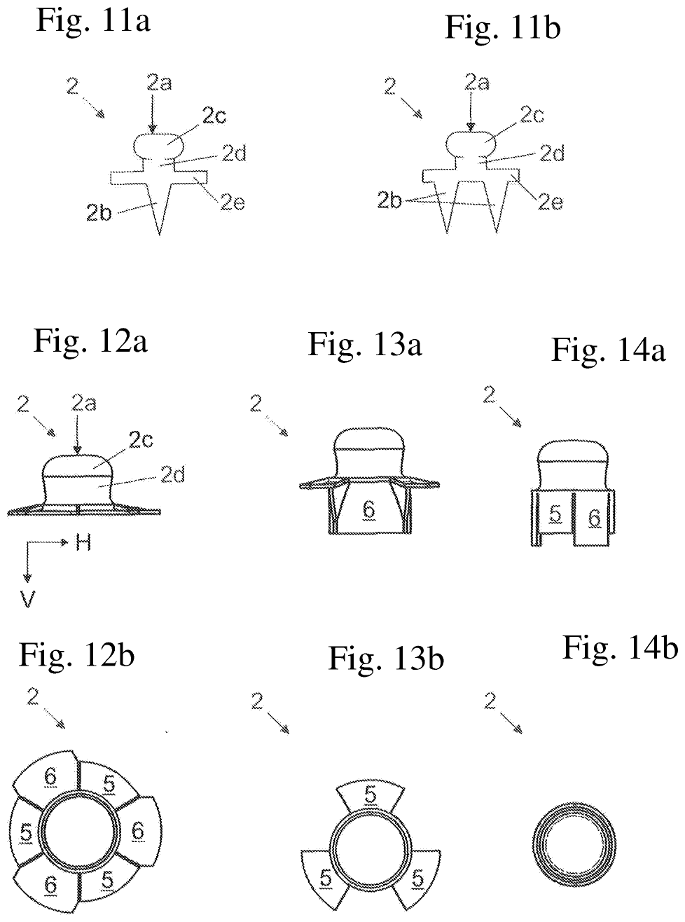

FIG. 11a shows an embodiment of a connecting element 2 in which the projection 2b is in the form of a tapering spike. In that way it is possible to introduce the connecting element 2 into the carrier 1 and the conductor 3 without previously producing a through opening 8 through the conductor 3 and the carrier 1. That therefore saves on a working step.

FIG. 11b shows an embodiment of a connecting element 2 in which there are two projections 2b in the form of tapering spikes. It will be noted however that there can be any number of projections 2b. In addition it is also possible to provide a plurality of projections 2b which are not of a spike shape. In that case the plurality of projections 2b can be arranged on the connecting element 2 in rotationally symmetrical or non-rotationally symmetrical relationship.

FIGS. 12a through 14b show embodiments of a connecting element 2 in which the projection and the flange-like holding region of the connecting element 2 are formed from at least one first segment 5 and at least one second segment 6.

It can also be seen that the second segments 6 are longer than the first segments 5. The segments 5, 6 can also be of equal length or the segments 5 can be longer than the segments 6.

FIG. 12a shows the connecting element in a front view when all segments 5, 6 are in a horizontal position H. FIG. 12b shows the corresponding plan view.

FIG. 13a shows a front view of the connecting element when the segments 5 are in a horizontal position H and the segments 6 are in a vertical position V. FIG. 13b shows the corresponding plan view.

FIG. 14a shows a front view of the connecting element when all segments 5, 6 are in a vertical position V. FIG. 14b shows the corresponding plan view.

In a connecting element 2 as shown in FIGS. 12a and 12b, prior to fitment of the connecting element 2 into the carrier 1, at least a first segment 5 of the at least two segments 5, 6 is moved into a vertical position V and after introduction of the connecting element 2 into the carrier 1 the at least one first segment 5 is moved into a horizontal position H again.

In an embodiment of a connecting element 2 as shown in FIGS. 14a and 14b before the connecting element 2 is fitted into the carrier 1 at least the one first segment 5 is moved into a horizontal position H while after the connecting element 2 is introduced into the carrier 1 at least one second segment 6 is moved into a horizontal position H.

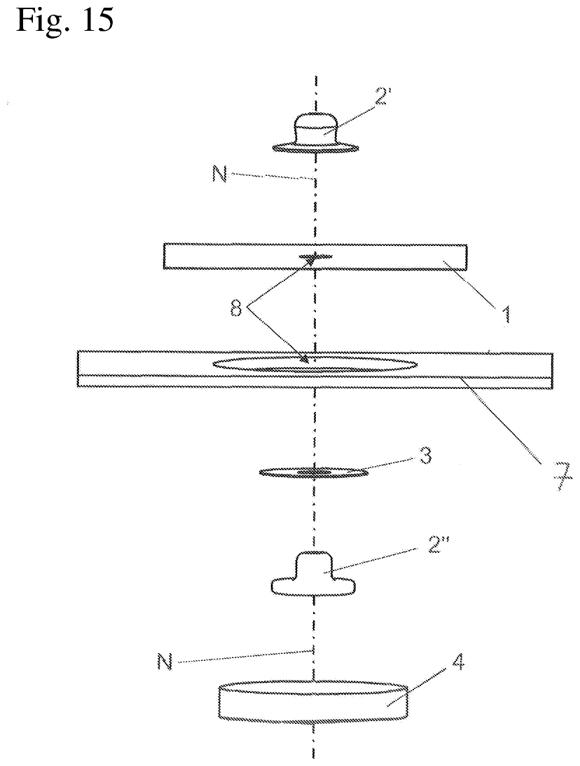

The further embodiment shown in FIG. 15 involves a central electrode with a two-part connecting element. The two parts of the connecting element 2 are denoted by references 2′ and 2″. For assembly purposes those two parts 2′, 2″ of the connecting element 2 are introduced from different sides into the opening 8, carrier 1 and conductor 3 and fitted together. They can be simply connected together in clamping relationship. There is however also the possibility of connecting those two parts 2′, 2″ in a different way—for example by clamping the neck together, by gluing, welding or soldering.

FIG. 15 further shows a notional normal N which extends on to the carrier 1 and which preferably extends centrally or centrically through the connecting element 2. That notional normal N also preferably extends centrically through the conductor 3 which is of a ring configuration (more precisely: through the opening in the conductor ring) and through the contact medium 4, there also preferably centrally. In this central electrode therefore the parts connecting element 2, conductor 3 and contact medium 4 are directly one below the other and are not laterally displaced relative to each other, preferably not at all as shown in FIG. 15 or only slightly so that the normal N always still passes through all three parts 2, 3 and 4 (or openings therein) or intersects same.

In relation to rotationally symmetrical components “centrically” means extending through the center point. In the case of non-rotationally symmetrical components “centrically” means extending through the center of gravity of the surface in plan view.

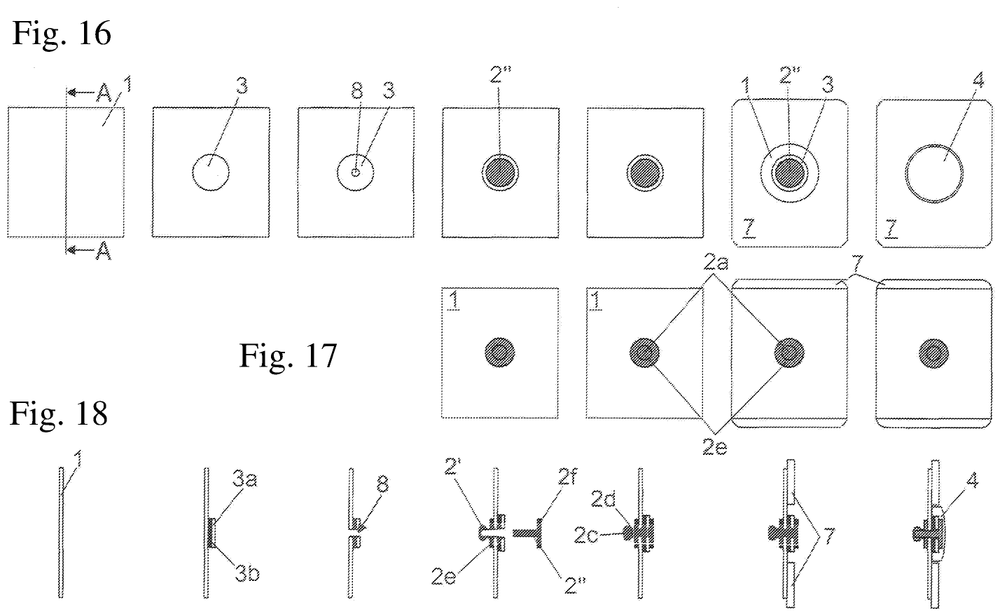

In the embodiment shown in FIGS. 16 through 18 most of the method steps are the same as those in FIGS. 1 through 3, for which reason identical references also denote the same parts.

The difference is essentially that this arrangement has a two-part connecting element 2. A first part 2′ and a second part 2″ are brought together from different sides of the carrier 1 and fitted together.

A lower holding region 2f of the second part 2″ of the connecting element 2 then bears against the carrier 1 or the conductor 3 and thus covers the side of the conductor 3, that later faces towards a contact medium 4. In addition that provides for making the galvanic connection between the contact element 2 and the conductor 3. That lower holding region 2f functionally substantially corresponds to the deformed enlarged region BZ of the preceding embodiments.

### LIST OF REFERENCES

1 carrier
2 connecting element
2′ first part of the connecting element 2
2″ second part of the connecting element 2
2a connecting location
2b projection
2c head
2d neck
2e holding region
2f lower holding region
3 conductor
3a electrically conducting material
3b electrically non-conducting material
4 contact medium
5 first segment
6 second segment
7 plaster layer
8 opening
9 wing segment
9a wing segment portion
11 adhesive layer (skin adhesive)
11a opening
H horizontal
N normal
V vertical
BZ deformed enlarged region

1. An electrode for application to the human skin having an electrically non-conducting carrier, wherein provided on a top side of the carrier that faces away from the skin is a projecting electrically conducting connecting element having a connecting location for releasable connection of a signal conductor, wherein there is provided a conductor which is arranged at least partially on the opposite underside of the carrier and which is electrically connected to the connecting element and to a contact medium that faces towards the skin, wherein the conductor and the contact medium are arranged on the underside of the carrier beneath the connecting location of the connecting element, wherein a notional normal to the carrier which extends—preferably centrically—through the connecting element extends—also preferably centrically—through the conductor or an opening in the conductor and the contact medium or an opening in the contact medium, wherein the conductor at its surface—preferably at the surface facing towards the skin—at least region-wise has silver/silver chloride or tin/tin chloride or another redox couple which is suitable for example for depolarization of the electrode, characterised in that the connecting element has at least one projection which extends through the carrier and which at its end has an enlarged region and that the conductor is arranged at least partially between the enlarged region and the carrier.

2. The electrode as set forth in claim 1, wherein the side of the conductor, that is towards the contact medium, is partially or completely covered by the connecting element.

3. The electrode as set forth in claim 1, wherein the region of the conductor having silver/silver chloride or tin/tin chloride or other redox couple suitable for depolarization of the electrode is electrically in contact with the connecting element and/or with the contact medium.

4. The electrode as set forth in claim 1, wherein the conductor is in the form of a layer comprising a first material which is provided, preferably coated, with a second electrically conducting material in the region of the contact medium.

5. The electrode as set forth in claim 4, wherein the second material is arranged in ring form on the first material.

6. The electrode as set forth in claim 4, wherein the second material is arranged substantially over the full surface area on the first material.

7. The electrode as set forth in claim 4, wherein the first material is a plastic, in particular a plastic film, or a conductor, for example of steel, wherein the second material is preferably silver/silver chloride, tin/tin chloride or another redox couple suitable for depolarization of the electrode.

8. The electrode as set forth in claim 4, wherein the first material is of a thickness of between 10 μm and 250 μm, preferably a thickness of between 30 μm and 100 μm, and the second material is of a thickness of between 0.05 μm and 30 μm, preferably a thickness of between 0.1 μm and 3 μm.

9. The electrode as set forth in claim 1, wherein the conductor is formed by a layer of a conductive material, that is applied to, preferably printed on, the carrier.

10. The electrode as set forth in claim 1, wherein the conductor is a metal or a metal alloy or a plastic film which for example is conducting throughout or at the surface by conductive carbon fibers or a textile material which is conducting throughout or at the surface.

11. The electrode as set forth in claim 1, wherein the conductor is of a substantially rotationally symmetrical, in particular ring-shaped, configuration.

12. The electrode as set forth in claim 1, wherein the conductor is of a substantially cuboidal configuration.

13. The electrode as set forth in claim 1, wherein the conductor has an opening for introduction of the connecting element.

14. The electrode as set forth claim 1, wherein the connecting element comprises metal, preferably a deep-drawn metal sheet, or conductive plastic, preferably ABS doped with conductive carbon fibers.

15. The electrode as set forth in claim 1, wherein the connecting element comprises at least two parts, wherein one of the two parts has the connecting location for releasable connection of a signal line.

16. The electrode as set forth in claim 15, wherein the at least two parts of the connecting element comprise the same or at least two different materials.

17. The electrode as set forth in claim 1, wherein the connecting element comprises a single part which has the connecting location for releasable connection of a signal line.

18. The electrode as set forth in claim 1, wherein the connecting element and/or the conductor and/or the contact medium is/are overall substantially rotationally symmetrical.

19. The electrode as set forth in claim 1, wherein the enlarged region is formed by deformation, wherein on the one hand an electrical connection between the conductor and the connecting element and on the other hand a mechanical fixing of the connecting element to the carrier can be made by that deformed enlarged region.

20. The electrode as set forth in claim 1, wherein the contact medium—preferably arranged in a recess in a plaster layer—is a gel preferably doped with chlorides, is in the form of a conductive adhesive, or is in the form of a sponge filled with saline solution.

21. The electrode as set forth in claim 1, wherein the connecting element is connected on the underside and/or the top side of the carrier to same—preferably with the interposition of a conductor of a planar configuration.

22. The electrode as set forth in claim 1, wherein a plaster layer is provided on an underside of the electrode that faces away from the skin, wherein the plaster layer can be glued on to the skin—preferably by means of a patient-side coating of biocompatible adhesive—in order to fix the electrode.

23. The electrode as set forth in claim 22, wherein the plaster layer can be glued to the carrier by way of a layer of pressure sensitive adhesive that is applied to the plaster layer or the carrier or a thermoactivatable adhesive.

24. The electrode as set forth in claim 1, wherein the carrier comprises a film, in particular of PET.

25. The electrode as set forth in claim 1, wherein the carrier is coated on the side towards the skin with adhesive, preferably a skin adhesive, which is preferably a pressure sensitive adhesive or is thermoactivatable, or has a plaster layer provided with an adhesive, preferably a skin adhesive.

26. The electrode as set forth in claim 1, wherein the connecting element at its surface is free from silver or silver/silver chloride and free from tin or tin/tin chloride and free from another redox couple—which is preferably suitable for depolarization of an electrode—or one of its components.

27. The electrode as set forth in claim 1, in the region of the connecting element the carrier has at least one incision which allows movability of the connecting element with respect to a plaster layer intended for adhesive bonding to the skin.

28. A method of producing an electrode for application to the human skin, in particular as set forth in claim 1, comprising:
arranging, preferably gluing or printing, a conductor on the underside, towards the skin, of an electrically non-conducting carrier,
introducing a connecting element through the carrier in such a way that the projection of the connecting element projects on the underside or the top side of the carrier and the connecting element bears against the top side or the underside of the carrier—preferably with a laterally projecting plate-shaped holding region, and
anchoring the connecting element in the carrier in such a way that an electrically conductive connection is made between the connecting element and the conductor and a mechanical fixing of the connecting element is made on the carrier.

29. The method as set forth in claim 28, wherein prior to introduction of a connecting element a through opening is made through the carrier and the conductor, preferably by stamping.

30. The method as set forth in claim 29, wherein anchoring of the connecting element is effected by deformation of the projection of the connecting element.

31. The method as set forth in claim 30, wherein deformation of the projection is effected by:
fusing of the projection, and/or
beading over of the projection, and/or
spreading the projection open, and/or
bending the projection over.

32. The method as set forth in claim 28, wherein anchoring of the connecting element is effected by connecting, preferably fitting together, a second part of the connecting element to a first part of the connecting element.

33. The method as set forth in claim 28, comprising the following further steps:
applying—preferably gluing—a plaster layer which is adhesive on the skin side to the carrier, and
introducing an electrical contact medium—preferably a gel—into the recess in the plaster layer in such a way that the subjacent conductor is contacted.

34. The method as set forth in claim 28, wherein prior to introduction of the connecting element the carrier is coated over its full surface area or part of its surface area with an adhesive, preferably a skin adhesive.

35. The method as set forth in claim 34, wherein after coating of the carrier with an adhesive, preferably a skin adhesive, a first contact medium is applied in such a way that the subjacent conductor is contacted.
